(19) United States
(12) Patent Application Publication
(10) Pub. No.: US 2021/0235900 A1
(43) Pub. Date: Aug. 5, 2021

(54) PILLOW FOR RELIEVING SLEEP APNEA AND PREVENTING SNORING

(71) Applicant: Sun Geuk CHOI, Chuncheon-si (KR)

(72) Inventor: Sun Geuk CHOI, Chuncheon-si (KR)

(21) Appl. No.: 16/973,413

(22) PCT Filed: Mar. 22, 2019
(86) PCT No.: PCT/KR2019/003339

§ 371 Date: Dec. 8, 2020

(30) Foreign Application Priority Data
Jun. 8, 2018 (KR) 10-2018-0066228

(51) Int. Cl. A47G 9/10; A61F 5/56

(52) U.S. Cl. CPC A47G 9/1081; A47G 9/1009; A61F 5/56

(57) ABSTRACT

The present invention relates to pillow for relieving sleep apnea and preventing snoring, the pillow comprising: a pillow body for supporting a sleeper's head and neck; and a support protrusion protruding from the center of the pillow body, wherein the support protrusion is configured by a curvature pattern portion so as to increase the area in contact with the sleeper's head, and the curvature pattern portion includes: a pair of first curved surface portions, each of which has a concave curved surface having a slope gradually increasing from a surface of the pillow body; and a second curved surface portion connecting the ends of the pair of first curved surface portions to each other and forming a convex curved surface having a slope gradually decreasing toward the center thereof. Therefore, the present invention can support an occipital region of a sleeper sleeping lying on his/her side, in more comfortable state, and thus can induce restful sleep accordingly.

### FIELD OF INVENTION

The present invention relates to a pillow for relieving sleep apnea and preventing snoring, and more particularly, to a pillow for relieving sleep apnea and preventing snoring, which stably supports the back of the head of a sleeper who sleeps while lying on their side and prevents the sleeper from lying on the back to thus guide the sleeper to have a deep sleep for a long time without snoring.

### BACKGROUND OF INVENTION

In general, snoring is caused by vibration of a wall in the neck occurring when air passes, since muscles around the neck and/or tongue often relax and the inner muscle of the tongue droops to narrow the airway during sleep. Especially, when sleeping while lying on the back or opening the mouth, the tongue muscle is prone to droop, thus causing snoring. That is, since snoring occurs due to an air path narrowed during sleep, the snorer has problems such as poor deep sleep or annoying other sleepers therearound due to a loud noise.

Recently, a conventional technique for prevention of snoring as shown in FIG. 1 has been disclosed, characterized in that the head of a sleeping person (“sleeper”) lying on their side is prevented from lying on the back, thereby preventing snoring.

That is, a snoring preventive (“anti-snoring”) pillow according to the prior art has a central protrusion 140 formed in the center portion so as to prevent the head of a sleeper sleeping on their side from lying face up.

However, the anti-snoring pillow according to the prior art has a configuration of the central protrusion 140 which is made of the same material as that of first and second head side supports 112 and 114, and simply protrudes at a predetermined height, so that the sleeper's head may easily ride over or press the central protrusion 140 by a motion of lying down unconsciously and thus being disposed in a front direction. Accordingly, there is a lack of functions for maintaining a stable posture to prevent snoring. Further, there is a disadvantage in that elasticity or cushioning of the central protrusion 14 is considerably deteriorated during prolonged use and thus the central protrusion 14 no longer perform as a support function to prevent snoring.

In addition, with regard to the conventional snoring preventive pillow, the first head side support 112 at one side and the second head side support 114 at the other side relative to the central protrusion 140 have a difference in height to a surface position, wherein, when the sleeper sleeping on their side with the head on the first head side support 112 rides over the central protrusion 140 and moves to the second head side support 114 unconsciously, a great head drop occurs due to the surface height and interferes with deep sleep, causing a problem such as waking from sleep.

### SUMMARY OF INVENTION

### Technical Problem to be Solved

The present invention has been designed to solve the above problems, and an object of the present invention is to develop a pillow for relieving sleep apnea and preventing snoring, which stably supports the back head of a sleeper sleeping while lying on their side and prevents lying on the back at the same time, whereby the sleeper can sleep deeply for a long time without snoring.

Further, the present invention provides a pillow for relieving sleep apnea and preventing snoring that can adjust the height of a support protrusion depending on physical conditions and sleep habits of the sleeper.

### Technical Solution

In order to accomplish the above objects, the pillow for relieving sleep apnea and preventing snoring according to the present invention may include: a pillow body to support the head and neck of a sleeper; and a support protrusion protruding from the center of the pillow body, wherein the support protrusion is formed of a curved pattern part to increase an area in contact with the head of the sleeper, and the curved pattern part includes: a pair of first curved portions having a curved concave surface with gradually increased slope from a surface of the pillow body; and a second curved portion which interconnects the distal ends of the pair of first curved portions and has a curved convex surface with gradually decreased slope toward the center portion.

An insert core may be provided inside the support protrusion to increase shape and strength of the support protrusion, and the insert core may include a horizontal member installed inside the pillow body, and a vertical member installed in a direction perpendicular to the horizontal member and disposed inside the support protrusion.

When looking at the pillow body from the side, both sides of the support protrusion and the insert core may be formed in a curved shape having gradually decreased width toward an upper side.

At one side of the pillow body and the support protrusion, a core entrance through which the insert core is detachably mounted may be formed.

Each of the support protrusion and the insert core may consist of a plurality of detachable assemblies in order to adjust a height thereof depending on physical conditions and sleeping habits of the sleeper.

The pillow body may include an inclined curved portion at which a thickness of the pillow increases from the center toward both ends thereof.

Further, a head support groove may be formed on a top surface of the pillow body to minimize a pressure applied to ears of the sleeper lying on their side to sleep while significantly suppressing flow of the sleeper's head.

### Effect of Invention

According to the present invention with the configurations as described above, the support protrusion may have a curved pattern part to increase an area in contact with the head of a sleeper so as to support the back of the head of the sleeper who sleeps while lying on their side, in a more comfortable state, thereby exhibiting effects of promoting comfortable sleep.

Further, the support protrusion may be provided with an insert core to increase shape and strength of the support protrusion therein, so as to prevent the shape and a structure of the support protrusion from being easily deformed and, in addition, to resist a predetermined pressure of the head of the sleeper to climb over the support protrusion. Even when the head of the sleeper overcomes the resistance of the insert core and is positioned on the support protrusion, the sleeper's head may not be held by the support protrusion but naturally guided to the opposite surface of the pillow body, thus accomplishing effects of inhibiting snoring caused by a posture of the sleeper with the head lying face up.

In addition, the support protrusion and the insert core may be formed in a configuration of plural detachable assemblies, thereby efficiently adjusting a height of the support protrusion depending on physical conditions and sleeping habits of a sleeper.

### BRIEF DESCRIPTION OF DRAWINGS

FIG. 1 is a perspective view illustrating a snoring-prevention pillow in the prior art.

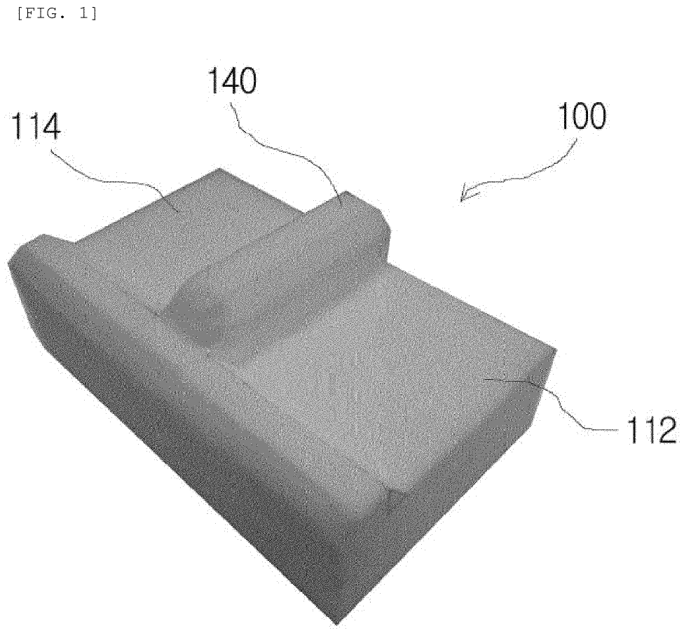

FIG. 2 is a perspective view illustrating a configuration of the pillow for relieving sleep apnea and preventing snoring according to an embodiment of the present invention.

FIG. 3 is a front view of FIG. 2.

FIG. 4 is an exploded perspective view of FIG. 2.

FIG. 5 is a prefabricated and exploded view illustrating another embodiment of the pillow for relieving sleep apnea and preventing snoring according to the present invention.

### DETAILED DESCRIPTION OF PREFERRED EMBODIMENTS OF INVENTION

Hereinafter, with reference to the accompanying drawings, preferred embodiments of the pillow for relieving sleep apnea and preventing snoring according to the present invention will be described in detail.

For reference, based on the principle that terms and words used in the present specification and claims should not be construed as being limited to common or dictionary meanings the inventors should properly define the concept of terms and words in order to explain their own invention by means of most preferred methods, the terms and words should be interpreted as having meanings and concepts corresponding to the technical spirit of the present invention.

Further, the configurations illustrated in the examples and drawings in the present specification are only the most preferred embodiments of the present invention and do not represent the whole technical spirit of the present invention and, therefore, it should be appreciated that various equivalents and modifications able to replace the present invention at the filing of the present application and thereafter may be included.

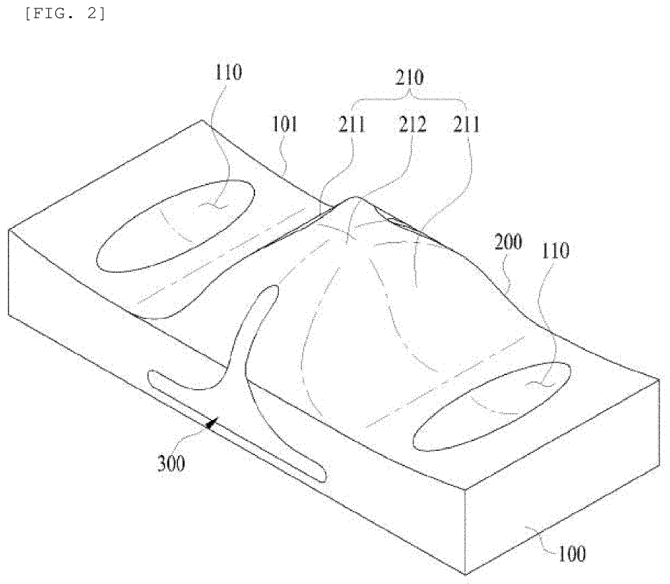

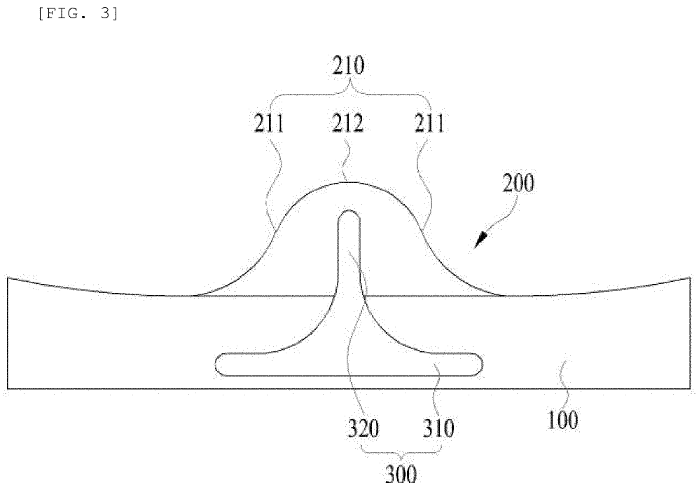

Hereinafter, referring to the accompanying FIG. 2 to FIG. 5, a preferred embodiment of the present invention will be described in detail.

As illustrated, the pillow for relieving sleep apnea and preventing snoring according to the present invention may include: a pillow body 100 to support the head and neck of a sleeper; and a support protrusion 200 protruding from the center of the pillow body 100.

First, the pillow body 100 has a configuration of comfortably supporting the head and neck of the sleeper who sleeps or lies down, wherein the pillow body 100 may be stitched after putting a cushioning material (cotton, sponge, etc.) with buffering properties therein, or may be made of latex or memory foam.

The pillow body 100 according to the present invention may include an inclined curved portion 101 at which a thickness of the pillow increases from the center toward both ends.

That is, a curved surface of the inclined curved portion 101 may have a functional role of suppressing the head of the sleeper lying on their side so as not to move or flow toward the outside of the pillow body 100.

Further, a head support groove 100 may be formed on a top surface of the pillow body 100 in order to significantly suppress flow of the head of a sleeper who sleeps while lying on their side and to minimize a pressure applied to ears of the sleeper, thus reducing burden thereto.

On the other hand, the support protrusion 200 is formed to protrude from the center of the pillow body 100, and serves to stably support the back of the head of the sleeper sleeping while lying on the their side.

That is, the support protrusion 200 may serve as a wall body to inhibit the head of the sleeper who sleeps while lying on their side from lying face up and thus prevent occurrence of snoring when sleeping face up.

Especially, the support protrusion 200 according to the present invention has a curved pattern part structure 210 that may induce comfortable contact by increasing an area in contact with the head of the sleeper.

More particularly, the curved pattern part 210 includes a pair of first curved portions 211 having a curved concave surface with gradually increased slope from a surface of the pillow body 100; and a second curved portion 212 which interconnects front ends of the pair of first curved portions 211 and has a curved convex surface with gradually decreased slope toward the center portion.

The curved pattern part 210 may support the back of the head of the sleeper who sleeps while lying on their side in a further comfortable state, thereby achieving effects of promoting comfortable sleep.

Further, when the pillow body 100 is viewed from the side, both sides of the support protrusion 200 may be formed in a curved shape with gradually reduced width in the upper direction (see FIG. 2).

Further, the configuration of the support protrusion 200 has an advantage of guiding the sleeper to freely and alternately use one side and the other side of the pillow body 100.

In addition, the support protrusion 200 may be provided with an insert core 300 to improve shape and strength of the support protrusion.

Herein, the insert core 300 includes a horizontal member 310 installed inside the pillow body 100, and a vertical member 320 installed in a direction perpendicular to the horizontal member 310 and disposed inside the support protrusion 200.

The insert core 300 is preferably made of rubber having high elastic strength so as to be elastically bent by an external pressure and then returned to an original position thereof, but is not limited thereto. Alternatively, the insert core may also be made of synthetic resin having high elastic strength.

In other words, the insert core 300 may be additionally provided in the support protrusion 200 to further increase the strength of the support protrusion 200, thus preventing shape and structure of the support protrusion 200 from being easily deformed. Further, the insert core may resist a predetermined pressure by which the head of a sleeper sleeping while lying on their side may climb over the support protrusion 200, thereby significantly preventing the sleeper from easily lying on their back (that is, face up). In addition, even when the head of the sleeper overcomes resistance of the support protrusion 200 and the insert core 300 and then is positioned on the support protrusion 200 due to turning force of the head in order to lie face up, the head of the sleeper cannot stay on the support protrusion 200 but naturally guided to the opposite surface of the pillow body by a curved structure of the support protrusion 200 and the elastic force of the insert core 300, whereby snoring caused by a posture of the head of the sleeper lying face up may be essentially inhibited.

Further, when the insert core 300 is viewed from the side of the pillow body 100, both sides of the insert core 300 are formed in a curved shape having gradually reduced width in the upper direction.

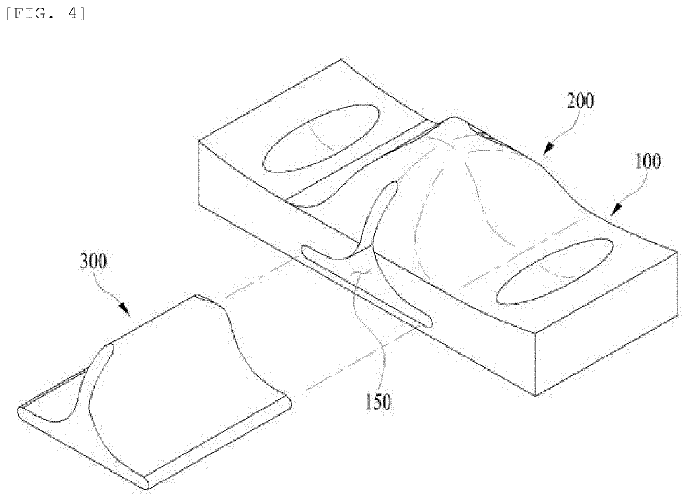

Further, as shown in FIG. 4, a core entrance 150 may be formed at one side of the pillow body 100 and the support protrusion 200, through which the insert core 300 is detachably provided.

That is, the insert core 300 is optionally provided or removed by the sleeper if needed.

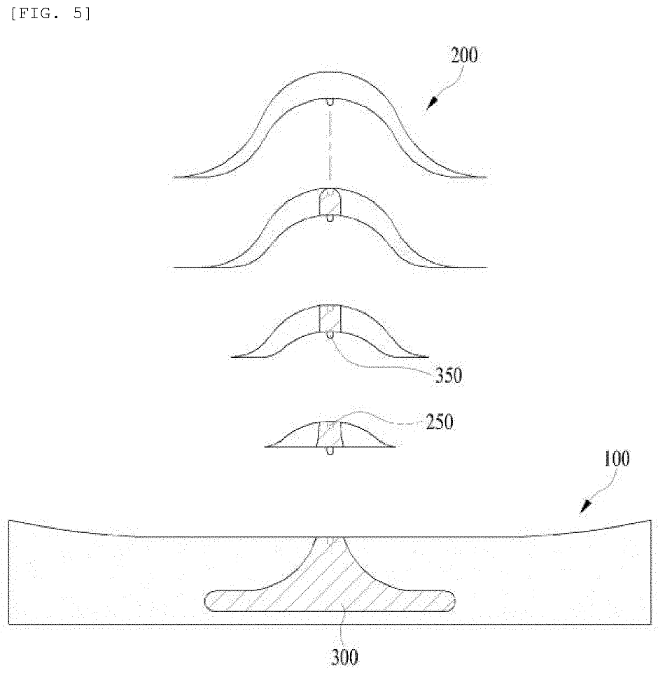

Further, as shown in FIG. 5, each of the support protrusion 200 and the insert core 300 may consist of a plurality of detachable assemblies to adjust a protruding height according to physical conditions and sleeping habits of the sleeper.

To this end, the support protrusion 200 and the insert core 300 may be divided into a plurality of assemblies, and such divided assemblies may be provided with fastening protrusions 350 and fastening grooves 250 detachably coupled to each other on the upper and lower portions of the divided assemblies.

Such an assembly configuration of the support protrusion 200 and the insert core 300 may effectively controls height of the support protrusion depending on physical structure and/or taste of the user, thereby attaining very useful effects in promoting comfortable sleep.

The present invention described above is not limited to the aforementioned embodiments and accompanying drawings, and it will be apparent to those skilled in the art that various substitutions, modifications and alterations are possible without departing from the technical spirit of the present invention.

1. A pillow for relieving sleep apnea and preventing snoring, comprising:
a pillow body to support the head and neck of a sleeper; and
a support protrusion protruding from the center of the pillow body,
wherein the support protrusion is formed of a curved pattern part to increase an area in contact with the head of the sleeper, and the curved pattern part includes: a pair of first curved portions having a curved concave surface with gradually increased slope from a surface of the pillow body; and a second curved portion which interconnects front ends of the pair of first curved portions and has a curved convex surface with gradually decreased slope toward the center portion.

2. The pillow according to claim 1, wherein an insert core is provided inside the support protrusion to increase shape and strength of the support protrusion, and the insert core includes: a horizontal member installed inside the pillow body; and a vertical member installed in a direction perpendicular to the horizontal member and disposed inside the support protrusion.

3. The pillow according to claim 2, wherein, when the pillow body is viewed from the side, both sides of the support protrusion and the insert core are formed in a curved shape having gradually decreased width toward an upper side.

4. The pillow according to claim 3, wherein a core entrance, through which the insert core is detachably mounted, is formed at one side of the pillow body and the support protrusion.

5. The pillow according to any one of claims 2 to 4, wherein each of the support protrusion and the insert core consists of a plurality of detachable assemblies in order to adjust a protruding height depending on physical conditions and sleeping habits of a sleeper.

6. The pillow according to claim 5, wherein an inclined curved portion, at which a thickness of the pillow increases from the center toward both ends thereof, is formed on the pillow body.

7. The pillow according to claim 6, wherein a head support groove is formed on a top surface of the pillow body in order to significantly suppress flow of the head of a sleeper who sleeps while lying on their side and to minimize pressure applied to ears of the sleeper, thus reducing burden thereto.
